(19) United States
(12) Patent Application Publication
(10) Pub. No.: US 2016/0235326 A1
(43) Pub. Date: Aug. 18, 2016

(54) A PORTABLE ELECTROMYOGRAPHIC SIGNAL MUSCLE RECUPERATION TREATMENT SYSTEM

(71) Applicant: HEALER TECH CO., LTD., Beijing (CN)

(72) Inventors: Shi CHENG, Beijing (CN); Li GE, Beijing (CN); Chen MIAO, Beijing (CN); Yifei REN, Beijing (CN)

(73) Assignee: HEALER TECH CO., LTD., Beijing (CN)

(21) Appl. No.: 15/026,591

(22) PCT Filed: Aug. 18, 2015
(86) PCT No.: PCT/CN2015/087368

§ 371 Date: Mar. 31, 2016

(30) Foreign Application Priority Data
Sep. 16, 2014 (CN) 2014205322524

(51) Int. Cl. A61B 5/0488; A61B 5/00; A61B 5/04; A61B 5/0492

(52) U.S. Cl. CPC A61B 5/0488; A61B 5/0492; A61B 5/486; A61B 5/04012

(57) ABSTRACT

The present invention provides a portable electromyographic signal neuromuscular rehabilitation system, comprising: a surface electromyography sensor, a signal transmitter, a rhythm generator, a comparator, and a, wherein the signal transmitter is connected to the surface electromyography sensor; the comparator is connected to the signal transmitter and the rhythm generator; the is connected to the rhythm generator and the comparator and configured to receive and display the specific rhythm to the user. The portable electromyographic signal neuromuscular rehabilitation system according to the present invention is portable and allows a remote monitoring. It enables a user to be treated at various locations. Besides, it can interestingly interact with the user, which avoids the tedium of lengthy treatment process, thereby enhancing the user's adherence to a long-term treatment and enables user to make self-treatment.

### FIELD OF THE INVENTION

The present invention relates to the field of medical device, and more specifically relates to a portable electromyographic signal neuromuscular rehabilitation system.

### BACKGROUND OF THE INVENTION

Biological feedback is a treatment concept of reflecting physiological activity conditions of a human body using various kinds of instruments, such that a user obtains a transient intuitive feeling, thereby achieving free control of physiological activities, which otherwise could not be voluntarily controlled, with an assistance of behavior stimulations of positive feedback and negative feedback. It has always been a leading-edge area of research how to apply biofeedback to enable a user to focus more on a treatment process and to adhere to a long-term treatment so as to achieve an optimal treatment outcome.

The existing neuromuscular rehabilitation system is generally used in a clinic or a hospital for rehabilitation, in which patients have to make an appointment and personally go to the facility to receive the treatment. Such a model wastes considerable time and manpower; besides, due to the tedium during the treatment process, the patient does not have a strong will to adhere to the long-term treatment. Consequently, it is less possible for patient's self-treatment. In addition, the existing neuromuscular rehabilitation system is not portable clue to its bulkiness and heavy dependence on wired USB transmission among the parts.

### SUMMARY OF THE INVENTION

To this end, the present invention provides a novel portable electromyographic signal neuromuscular rehabilitation system that may solve at least a part of the above problems.

The present invention provides a portable electromyographic signal neuromuscular rehabilitation system, comprising: a surface electromyography sensor, a signal transmitter, a rhythm generator, a comparator, and a renderer, wherein the surface electromyography sensor is configured to collect electromyographic signal of a user; the signal transmitter is connected to the surface electromyography sensor and configured to receive and transmit the electromyographic signal; the rhythm generator is configured to generate a specific rhythm; the comparator is connected to the signal transmitter and the rhythm generator and configured to receive the electromyographic signal and the specific rhythm, and compares the electromyographic signal and the specific rhythm to determine whether they match each other or not; the renderer is connected to the rhythm generator and the comparator and configured to receive and render the specific rhythm to the user, to provide a positive feedback when the electromyographic signal matches the specific rhythm and provide a negative feedback when the electromyographic signal does not match the specific rhythm.

Alternatively, the portable electromyographic signal neuromuscular rehabilitation system according to the present invention further comprises a signal processor, the signal processor is connected between the surface electromyography sensor and the signal transmitter.

Alternatively, in the portable electromyographic signal neuromuscular rehabilitation system according to the present invention, the surface electromyography sensor comprises an electrode sensor, a signal amplification circuit connected to the electrode sensor, a signal full-wave rectification circuit connected to the signal amplification circuit, and a signal smoothing circuit connected to the signal full-wave rectification circuit.

Alternatively, in the portable electromyographic signal neuromuscular rehabilitation system according to the present invention, the electrode sensor comprises a reference electrode, a muscle middle-end electrode, and a muscle terminal-end electrode.

Alternatively, in the portable electromyographic signal neuromuscular rehabilitation system according to the present invention, the signal processor comprises an A/D converter and a digital signal processor, the A/D converter being connected to the digital signal processor.

Alternatively, the portable electromyographic signal neuromuscular rehabilitation system according to the present invention further comprises a remote monitor, the remote monitor being connected to the renderer and configured to receive a feedback result from the renderer.

Alternatively, in the portable electromyographic signal neuromuscular rehabilitation system according to the present invention, the number of the electrode sensor is at least one.

Alternatively, in the portable electromyographic signal neuromuscular rehabilitation system according to the present invention, the signal transmitter is a wireless transmitter.

The portable electromyographic signal neuromuscular rehabilitation system according to the present invention is portable and enables a doctor or a therapist to monitor remotely. It facilitates a user to be treated at more places. Besides, the posture of the body is freer during the treatment. Further, it can interestingly interact with the user, which avoids the tedium of lengthy treatment process, thereby enhancing the user's adherence to a long-term treatment.

### BRIEF DESCRIPTION OF THE ACCOMPANYING DRAWINGS

By reading the following detailed description of preferred embodiments, It will be much easier for general technician in this field to understand other advantages and benefits. The drawings are only for the purpose of illustrating preferred embodiments and should not be regarded as limitations to the present invention. Moreover, in the whole drawings, the same reference numbers are used for representing the same components. In the accompanying drawings, alphabetical labels after the reference numbers represent a plurality of same components; in general when they generally refer to these components, their last alphabetic labels will be omitted. In the drawings:

FIG. 1 shows a structural diagram of a portable electromyographic signal neuromuscular rehabilitation system according to the present invention;

FIG. 2 shows a structural diagram of a surface electromyography sensor;

FIG. 3 shows a structural diagram of a signal processor;

Wherein the meanings of respective reference numerals in the drawings are specified below:

a surface electromyography sensor 10, an electrode sensor 11, a signal amplification circuit 12, a signal full-wave rectifying circuit 13, a signal smoothing circuit 14, a signal processor 15, an A/D converter 15-1, a digital signal processor 15-2, a signal transmitter 20, a rhythm generator 30, a comparator 40, a renderer 50, and a remote monitor 60.

### DETAILED DESCRIPTION OF THE PREFERRED EMBODIMENTS

Hereinafter, the present utility mode will be further described in conjunction with the accompanying drawings and preferred embodiments.

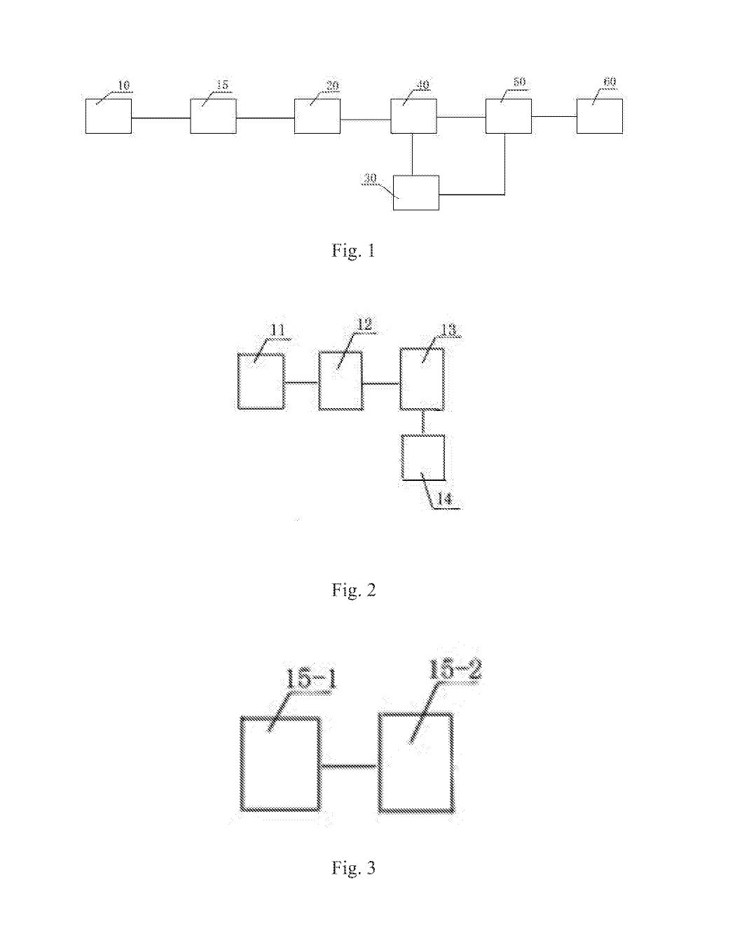

FIG. 1 shows a structural diagram of a portable electromyographic signal neuromuscular rehabilitation system according to the present invention. As shown in FIG. 1, the portable electromyographic signal neuromuscular rehabilitation system comprises: a surface electromyography sensor 10, a signal processor 15, a signal transmitter 20, a rhythm generator 30, a comparator 40, and a renderer 50, wherein the surface electromyography sensor 10 is configured to collect electromyographic signal of a user; and the signal transmitter 20 is connected to the surface electromyography sensor 10. The connection between the signal transmitter 20 and the surface electromyography sensor 10 may be a direct connection or an indirect connection. For the indirect connection manner, the signal transmitter 20 is connected to the signal transmitter 20 via the signal processor 15; and the signal processor 15 is disposed between the surface electromyography sensor 10 and the signal transmitter 20. The rhythm generator 30 is configured to generate a specific rhythm; the comparator 40 is connected to the signal transmitter 20 and the rhythm generator 30. The comparator 40 is for receiving the electromyographic signal and the specific rhythm, and comparing the electromyographic signal with the specific rhythm to determine whether they match each other or not. The renderer 50 is connected to the rhythm generator 30 and the comparator, is configured to receive and render the specific rhythm to the user, provides a positive feedback when the electromyographic signal matches the specific rhythm, and provides a negative feedback when the electromyographic signal does not match the specific rhythm.

The signal transmitter 20 is connected to the comparator 40; the comparator 40 and the rhythm generator 30 are connected to the renderer 50. The renderer 50 is a smart phone, a smart tablet, a computer, a PDA, a smart wearable device (e.g., a smart watch, a smart band, a smart glass), etc. The comparator 40 receives the electromyographic signal and the specific rhythm generated by the rhythm generator 30; and then compared the electromyographic signal with the specific rhythm to determine whether they match each other or not. The renderer 50 is for receiving and rendering the specific rhythm to the user, and providing a positive feedback when the electromyographic signal matches the specific rhythm, or a negative feedback when the electromyographic signal does not match the specific rhythm. With a music playing application as an example, the rhythm generator 30 defines the rhythm prompt shown based on the rhythm of playing the music as the specific rhythm. The user reacts based on the rhythm prompt rendered by the renderer 50. The reaction is transmitted to the comparator 40. The reaction on the comparator 40 is compared to the rhythm prompt of the rhythm generator 30. In the case of matching, the renderer 50 provides a positive feedback, e.g., a “tick tock” sound or a signal with a red light; in the case of not matching, the renderer 50 provides a negative feedback, e.g., a “buzz” sound or a signal with a yellow light. With Parkour game application as another example, the rhythm generator 30 defines various obstacles in the Parkour game application as specific rhythms. The user responds to avoid the obstacles according to various obstacles rendered by the renderer 50. The response is transmitted to the comparator 40. The response on the comparator 40 is compared to a specific rhythm of the rhythm generator 30 (i.e., various obstacles). In the case of matching, the renderer provides a positive feedback, e.g., a “tick tock” sound or a signal with red light; in the case of not matching, the renderer 50 provides a negative feedback, e.g., a “buzz” signal or a signal with yellow light. The portable electromyographic signal neuromuscular rehabilitation system enables the user to constantly strengthen a target muscle or muscle group through the rhythm generator 30, comparator 40, and renderer 50, and skillfully exercise voluntary control of the target muscle by facilitating the motor nerve control and suppressing improper contraction of the muscle so as to achieve muscle balance; meanwhile, It is characterized by its high interactivity, intelligence and entertainment, which enhances the user's will to adhere to the long-term treatment, and makes user's self-treatment possible.

In the portable electromyographic signal neuromuscular rehabilitation system, the portable electromyographic signal neuromuscular rehabilitation system further comprises a signal processor 15. As shown in FIG. 1, the signal processor 15 is connected between the surface electromyography sensor 10 and the signal transmitter 20. The surface electromyography sensor 10 collects an electromyographic signal from a body surface. The electromyographic signal is amplified, converted, smoothed, and square waved to derive a second electrical signal; the second electrical signal is transmitted to the signal processor 15; the signal processor is connected to the signal transmitter 20. The signal processor 15 converts the second electrical signal into a first digital signal. By taking the algorithm of numerical integration and averaging, a second digital signal can be derived from the first digital signal. Then, the second digital signal is transmitted to the signal transmitter 20.

In the portable electromyographic signal neuromuscular rehabilitation system, the surface electromyography sensor signal collector 10 comprises an electrode sensor 11, a signal amplifier 12, a signal full-wave rectification circuit 13, and a signal smoothing circuit 14. As shown in FIG. 2, the surface electromyography sensor signal collector 10 comprises an electrode sensor 11, a signal amplifying circuit 12, a signal full-wave rectification circuit 13, and signal smoothing circuit 14. The electrode sensor 11 is connected to the signal amplifying circuit 12. The electrode sensor 11 comprises a reference electrode, a muscle middle-end electrode, and a muscle terminal-end electrode. The electrode sensor 11 transmits a first electrical signal obtained from the body surface to the signal amplifying circuit 12. The signal amplifying circuit 12 amplifies the first electrical signal and transmits it to the signal full-wave rectification circuit 13. The signal full-wave rectification circuit 13 is connected to the signal smoothing circuit 14. The signal full-wave rectification circuit 13 converts the alternative current into direct current, and transmits the direct current signal to the signal smoothing circuit 14. The signal smoothing circuit 14 smoothes the direct current signal and processes the square wave conversion to derive a second electrical signal which is transmitted to the signal processor 15.

In the portable electromyographic signal neuromuscular rehabilitation system, the signal processor 15 comprises an A/D converter 15-1 and a digital signal processor 15-2, the A/D converter 15-1 being connected to the digital signal processor 1.5-2. As shown in FIG. 3, the signal processor 15 comprises an A/D converter 15-1 and a digital signal processor 15-2, the A/D converter 15-1 is connected to the digital signal processor 15-2. The A/D converter 15-1 converts the second electrical signal transmitted from the above signal smoothing circuit 14 into a first digital signal, and then transmits the first digital signal to the digital signal processor 15-2. The digital signal processor 15-2 inplement the algorithm of numerical integration and averaging algorithm processing to the first digital signal to derive a second digital signal, and then transmits the second digital signal to the signal transmitter 20.

In the portable electromyographic signal neuromuscular rehabilitation system, there further comprises a remote monitor 60. The remote monitor 60 is connected to the renderer 50. The feedback result generated by interaction with the user and obtained by the renderer 50 is wirelessly transmitted to the remote monitor 60. The doctor may monitor the user's muscle activity based on the feedback result on the remote monitor 60, and thereby formulating different therapy plan.

In the portable electromyographic signal neuromuscular rehabilitation system, the number of the electrode sensor 11 is at least one. The number of the electrode sensor 11 is at least one, and the more the number of the electrode sensor 11 is, the more the electrical signals are collected from the surface body, the better the treatment is handled.

In the portable electromyographic signal neuromuscular rehabilitation system, the signal transmitter is a wireless transmitter. The wireless transmitter is a Blue tooth, an infrared transmitter or WiFi. The most important property of Bluetooth is power saving; Because the Bluetooth power consumption of running and standby is extremely low, a piece of button cell is able to continuously work for several years supporting a Bluetooth device. The main advantages thereof are: very low peak value, average and standby mode power consumption, low cost, enhanced wireless coverage, complete downward compatibility and low delay (APT-X). The signal transmitter is preferably Bluetooth.

The portable electromyographic signal neuromuscular rehabilitation system according to the present invention is portable and enables a doctor or a therapist to monitor remotely. It facilitates a user to be treated at more places. Besides, the posture of the body is freer during the treatment. It can interestingly interact with the user, which avoids the tedium of lengthy treatment process, thereby enhancing the user's adherence to a long-term treatment.

It should be noted that the above embodiments intend to illustrate, rather than limit, the present invention. Moreover, without departing from the scope of the appended claims, those skilled in the art may design alternative embodiments. In the claims, no reference numerals included in the parentheses should constitute limitations to the claims. The word “comprise” should not exclude the elements or steps not listed in the claims. The word “a” or “one” before an element does not exclude a plurality of such elements. The present invention may be implemented by hardware including several different elements and an appropriately programmed computer. In the claim with several modules, some of the modules may be specifically embodied by one hardware device. Using of words such as first, second, and third does not represent any sequence. These words may be interpreted as names.

1. A portable electromyographic signal neuromuscular rehabilitation system, comprising: a surface electromyography sensor, a signal transmitter, a rhythm generator, a comparator, and a renderer, wherein
the surface electromyography sensor is configured to collect electromyographic signal of a user;
the signal transmitter is connected to the surface electromyography sensor and configured to receive and transmit the electromyographic signal;
the rhythm generator is configured to generate a specific rhythm;
the comparator is connected to the signal transmitter and the rhythm generator and configured to receive the electromyographic signal and the specific rhythm, and compares the electromyographic signal with the specific rhythm to determine whether they match each other or not;
the renderer is connected to the rhythm generator and the comparator and configured to receive and display the specific rhythm to the user, and provides a positive feedback when the electromyographic signal matches the specific rhythm or provides a negative feedback when the electromyographic signal does not match the specific rhythm.

2. The portable electromyographic signal neuromuscular rehabilitation system according to claim 1, further comprising a signal processor connected between the surface electromyography sensor and the signal transmitter.

3. The portable electromyographic signal neuromuscular rehabilitation system according to claim 1, wherein the surface electromyography sensor signal collector comprises an electrode sensor, a signal amplification circuit connected to the electrode sensor, a signal full-wave rectification circuit connected to the signal amplification circuit, and a signal smoothing circuit connected to the signal full-wave rectification circuit.

4. The portable electromyographic signal neuromuscular rehabilitation system according to claim 3, wherein, the electrode sensor comprises a reference electrode, a muscle middle-end electrode, and a muscle terminal-end electrode.

5. The portable electromyographic signal neuromuscular rehabilitation system according to claim 1, wherein the signal processor comprises an A/D converter and a digital signal processor connected to the A/D converter.

6. The portable electromyographic signal neuromuscular rehabilitation system according to claim 1, further comprising a remote monitor connected to the renderer and configured to receive feedbacks from the renderer.

7. The portable electromyographic signal neuromuscular rehabilitation system according to claim 1, wherein the number of the electrode sensor is at least one.

8. The portable electromyographic signal neuromuscular rehabilitation system according to claim 1, wherein the signal transmitter is a wireless transmitter.
